(19) United States
(12) Patent Application Publication
(10) Pub. No.: US 2007/0123989 A1
(43) Pub. Date: May 31, 2007

(54) Method and instruments to treat spondylolisthesis by an anterior minimally invasive approach of the spine

(75) Inventors: Andy Gfeller, Rohr (CH); Remo Amherd, Ramlinsburg (CH); Thierry Stoll, Meinisberg (CH)

Correspondence Address: JONES DAY, 222 EAST 41ST STREET, NEW YORK, NY, 10017-6702, US

(73) Assignee: Synthes (U.S.A.)

(21) Appl. No.: 11/583,745

(22) Filed: Oct. 18, 2006

Related U.S. Application Data
(60) Provisional application No. 60/728,919, filed on Oct. 21, 2005.

(51) Int. Cl. A61F 2/44

(52) U.S. Cl. 623/17.16

(57) ABSTRACT

A method for intra-operative surgical treatment of spondylolisthesis by an anterior minimally invasive approach of the lumbar spine includes inserting an interbody spacer between two vertebrae, attaching an anatomically designed reduction plate to at least one of the two vertebrae, and attaching the interbody spacer to the reduction plate by a fastening means through a central borehole of the reduction plate and the interbody spacer. The interbody spacer may be attached to the anteriorly positioned vertebra by at least bone screw. The upper and lower parts may be attached to the upper and lower vertebra by at least one bone screw to stabilize the displaced vertebral segment of the spine.

### TECHNICAL FIELD

This application is related to U.S. Provisional Patent Application No. 60/728,919, entitled “METHOD AND INSTRUMENTS TO TREAT SPONDYLOLISTHESIS BY AN ANTERIOR MINIMALLY INVASIVE APPROACH OF THE SPINE”, filed Oct. 21, 2005, which is incorporated by reference herein in its entirety.

### TECHNICAL FIELD

The invention relates to methods and instruments that may be used for intra-operative surgical treatment of spondylolisthesis by an anterior minimally invasive approach of the spine.

### BACKGROUND OF THE INVENTION

Spondylolisthesis is a term used to describe when one vertebra slips forward on the vertebra below it (FIG. 1). This usually occurs because there is a spondylolysis in the superior vertebra. There are two main parts of the spine that keep the vertebrae aligned, the disc and the facet joints. When spondylolysis occurs, the facet joint can no longer hold the vertebra back. The intervertebral disc may slowly stretch under the increased stress and allow the upper vertebra to slide forward. In the vast majority of cases, stretching of the intervertebral disc only allows for a small amount of forward slip.

Surgical treatment for spondylolisthesis needs to address both the mechanical symptoms and the compressive symptoms if they are present. Usually this means that the nerves exiting the spine should be freed of pressure and irritation. Performing a complete laminectomy (removing the lamina) usually accomplishes relieving the pressure and irritation on the nerves exiting the spine. Removing the lamina allows more room for the nerves. It also enables the surgeon to remove the lump of tissue surrounding the spondylolysis defect. The result is reduced irritation and inflammation on the nerves. Once the nerves are freed, a spinal fusion is usually performed to control the segmental instability. (source: www.spineuniversity.com)

The goals of surgery are to remove pressure on spinal nerves (i.e., decompression) and to provide stability to the lumbar spine. In most cases of spondylolisthesis, lumbar decompression should be accompanied by uniting one spinal vertebra to the next (i.e. spinal fusion) with spinal instrumentation (i.e., implants that are often used to help aid the healing process). Surgery can be performed from the back of or posterior approach to the spine (i.e., distraction and reduction can be achieved before tightening the posterior fixation) and/or from the front or an anterior approach to of the spine (i.e., anterior fusion). Such methods negatively affect the vital posterior muscular structures.

### SUMMARY OF THE INVENTION

The present invention provides a a method of performing spondylolisthesis reduction. Preferably the method, instruments and implants preserve the vital posterior muscular structures, thus reducing the surgical morbidity associated with fusion surgery, preferably including lumbar fusion surgery.

The method includes the steps of inserting an interbody spacer between two vertebrae, and attaching an anatomically designed reduction plate to the two vertebrae by two screws. The reduction plate includes upper and lower boreholes, where at least one screw, using a stable plate-screw connection, is fixed into the vertebra which is more anteriorly positioned than the other vertebra, and the other screw, a non-locking screw, is fixed to the other vertebra. The method further includes driving the non-locking screw to reduce the vertebral slippage distance.

In another embodiment, the method includes the steps of inserting an interbody spacer between two vertebrae, attaching an anatomically designed reduction plate to at least one of the two vertebrae using at least one non-locking screw, and attaching the interbody spacer to the reduction plate by a fastening means through a borehole, preferably a central borehole, of the reduction plate and the interbody spacer. The interbody spacer may be attached to the anteriorly positioned vertebra by at least one bone screw. The method further includes rotating the non-locking screw to reduce the vertebral slippage distance.

In still another embodiment, the method includes inserting an interbody spacer between two vertebrae, where the interbody spacer may be attached to the vertebrae by locking screws. The method further includes inserting a locking screw mechanism, and adjusting the locking screw mechanism such that the vertebrae are aligned vertically, wherein the superior or upper vertebra is moved in relation to the inferior or lower vertebra.

In a further embodiment, the method includes attaching pedicle screws to vertebrae surrounding a vertebra exhibiting a spondylolisthesis condition, attaching preassembled pedicle screws into the vertebra exhibiting the spondylolisthesis condition, and attaching rods to the pedicle screws. The method further includes repositioning the vertebra exhibiting the spondylolisthesis condition using a reduction instrument such that the head of the preassembled pedicle screws coincide with the rods, and affixing the preassembled pedicle screws to the rods using locking caps and a screwdriver.

In still a further embodiment, the method includes inserting a screw into the anterior area of adjacent vertebrae, where one of the adjacent vertebrae exhibits a spondylolisthesis condition, and fixing the screw attached to the vertebra not exhibiting the spondylolisthesis condition to an external rigid element. The method further includes using an adjustable mechanism to adjust the screw inserted into the vertebra exhibiting the spondylolisthesis condition until slippage distance of that vertebra is reduced. This method may be performed externally from an incision area.

Other objectives and advantages in addition to those discussed above will become apparent to those skilled in the art during the course of the description of a preferred embodiment of the invention which follows. In the description, reference is made to accompanying drawings, which form a part thereof, and which illustrate an example of the invention. Such example, however, is not exhaustive of the various embodiments of the invention, and the claims that follow should not be limited to the examples shown.

### BRIEF DESCRIPTION OF THE DRAWINGS

The spondylolisthesis reduction methods and instrumentation are explained in even greater detail in the following exemplary drawings, wherein the instrumentation and methods of operation may be better understood and wherein like references numerals represent like elements. The drawings are merely exemplary to illustrate the structure, operation and method of treating spondylolisthesis and certain features that may be used singularly or in combination with other features and the invention should not be limited to the embodiments shown.

FIG. 1 depicts a segment of a spine where one vertebra disc has moved or slipped forward of the other vertebrae (spondylolisthesis);

FIG. 2 is a side view of an embodiment of the present invention;

FIG. 3 is a side view of a modification of the embodiment depicted in FIG. 2;

FIG. 4A is a side view of another embodiment of the present invention;

FIGS. 4B and 4C are side views of the before and after positions of the implant surfaces of the embodiment depicted in FIG. 4A;

FIGS. 5A and 5B are perspective views of different embodiments of the implant of FIGS. 4A-C;

FIGS. 6A and B are side views of another embodiment of the present invention;

FIGS. 7A-D are side views of a reduction instrument;

FIGS. 8A and B are side views of another embodiment;

FIGS. 9A-D are side views of another embodiment; and

FIGS. 10A and 10B are views of another embodiment.

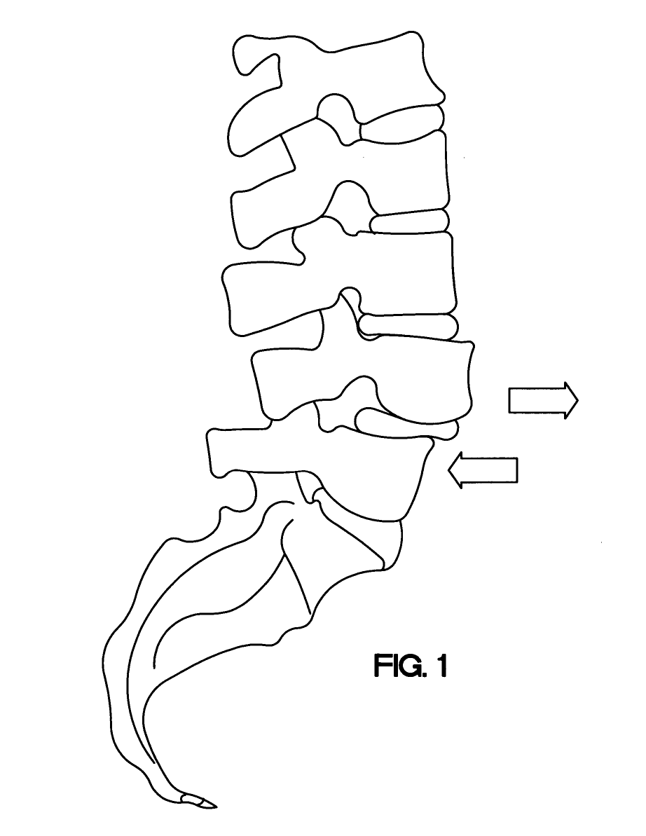

### DESCRIPTION OF THE PREFERRED EMBODIMENTS

Spondylolisthesis reduction can be performed either within the wound site (in situ) or outside the wound site (ex situ), where the wound site refers to the area of incision.

The in-situ methods allow for spondylolisthesis reduction by a minimally invasive approach, preferably using an implant as a reduction device.

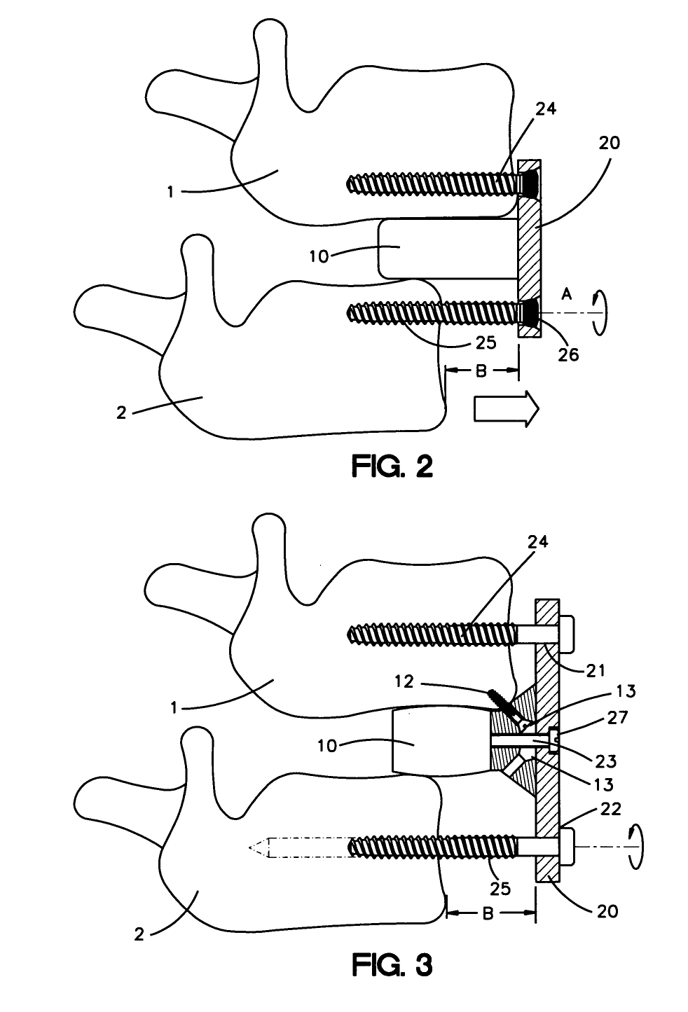

In a preferred embodiment, as depicted in FIG. 2, an interbody spacer 10 (e.g. metallic, allograft or polymeric cage) may be placed between the two vertebrae 1, 2. An anatomically designed reduction plate 20 with upper and lower borehole(s) may be placed in front of the treatment segment (site) and attached to the vertebrae by at least one upper screw 24 and at least one lower screw 25. The reduction plate 20 may either be straight or curved (pre-stressed). By fixing one screw 24 into the vertebra positioned more anteriorly by a stable or locking plate-screw connection 26 (LCP locking screw concept), the other non-locking screw 25 may be used to reduce the vertebral slipping distance B by driving the screw in a direction A, as depicted in FIG. 2. This is accomplished by a “lagging feature” which occurs when the head of the screw 25 comes in contact with the reduction plate 20, further turning of screw 25 causes the displaced vertebra to move posteriorly and align vertically with the other vertebrae.

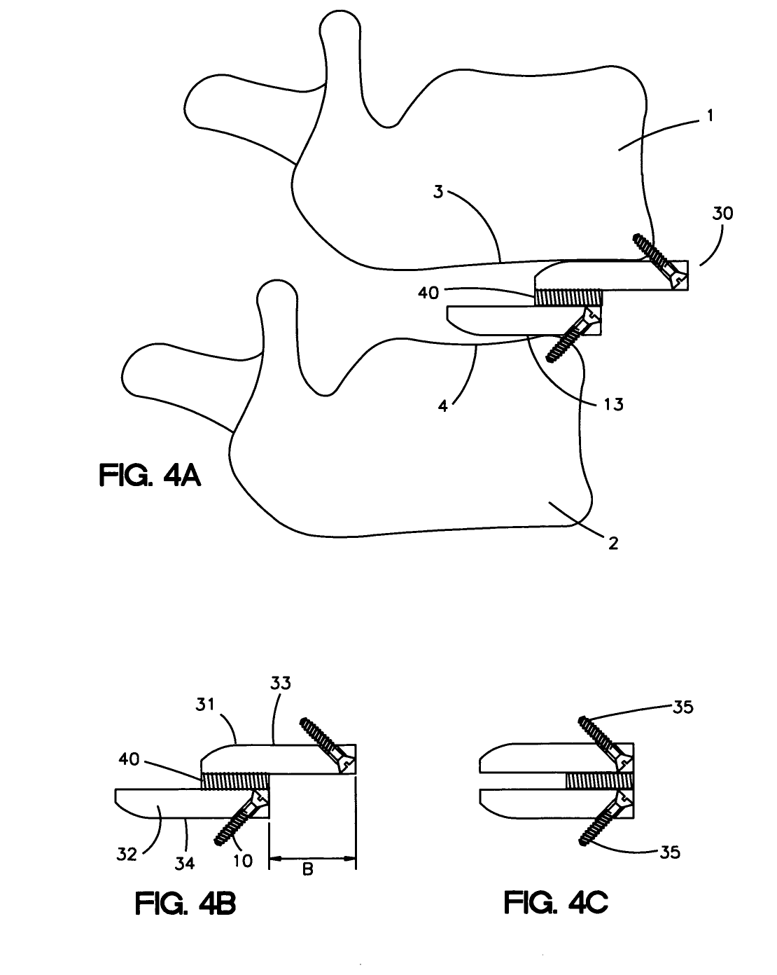

In an alternative configuration (FIG. 3) of the embodiment depicted in FIG. 2, the reduction plate 20 may be affixed by at least one screw 25 to the vertebra 2 that has slipped forward, and optionally a second screw 24 may be connected to the other vertebra 1. Further, the interbody spacer 10 may preferably be connected to the reduction plate 20, and is preferably expandable in height. The reduction plate 20 may have a central borehole 23 provided, preferably, with an internal thread (not shown), and preferably parallel to the upper and lower boreholes 21, 22. Correspondingly thereto, the interbody spacer 10 may have a central borehole 23 with an internal thread (not shown) for accommodating a fastening means 27, for example a screw, for fastening the reduction plate 20 to the interbody spacer 10. The interbody spacer 10 may have additional boreholes 13 such that the axes of these boreholes 13 are not parallel to each other or the central borehole 23. From the front surface of the interbody spacer 10, the additional boreholes may diverge. At least one longitudinal fixation element 12, for example a bone screw, may be used to further connect the interbody spacer 10 to the vertebra 1, thereby increasing the rotational stability of the reduced segment. The non-locking screw 25 may be used to reduce the vertebral slippage B by rotating the screw in a similar manner as in the embodiment depicted in FIG. 2.

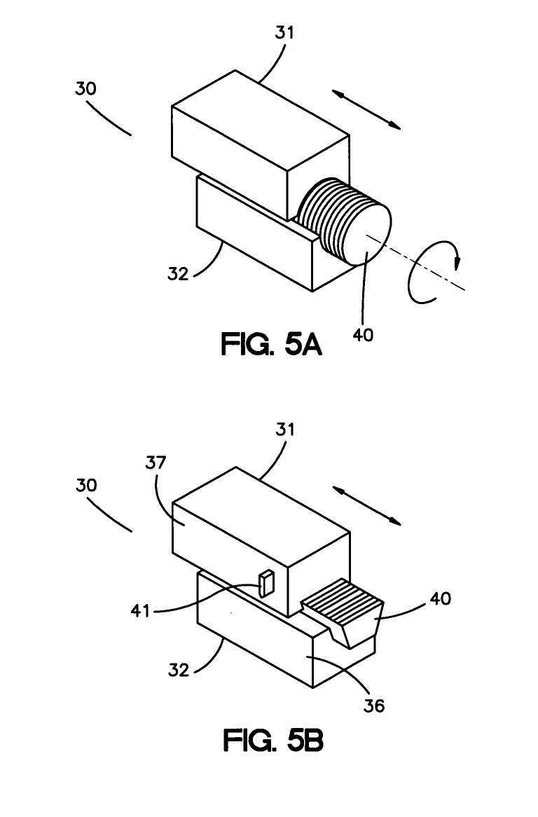

In another preferred embodiment, an interbody spacer 30, (e.g. SynCage, SynFix) is depicted in FIGS. 4A-C. The interbody spacer 30 may comprise two horizontal halves consisting of an upper half 31 and a lower half 32. The interbody spacer 30 may be placed between two vertebrae 1, 2, so that the contact surface 33 of the upper half 31 and the contact surface 34 of the lower half 32 fit the curvature of the upper 3 and lower 4 endplates of the vertebrae, respectively. The upper half 31 and lower half 32 of the interbody spacer 30 are fixed to the adjacent vertebrae with a locking screw mechanism 40. The interbody spacer 30 may further be attached to the adjoining upper and lower vertebrae 1, 2 by corresponding locking screws 35.

The slipping distance B of one vertebra can then be reduced by the locking screwing mechanism 40 that brings the interbody spacer halves 31, 32 into vertical alignment with each other, and thus realign the spine, as shown in FIG. 4C. The locking screwing mechanism 40 preferably moves the lower half 32 of the interbody spacer 30 with respect to the upper half 31 of the interbody spacer 30.

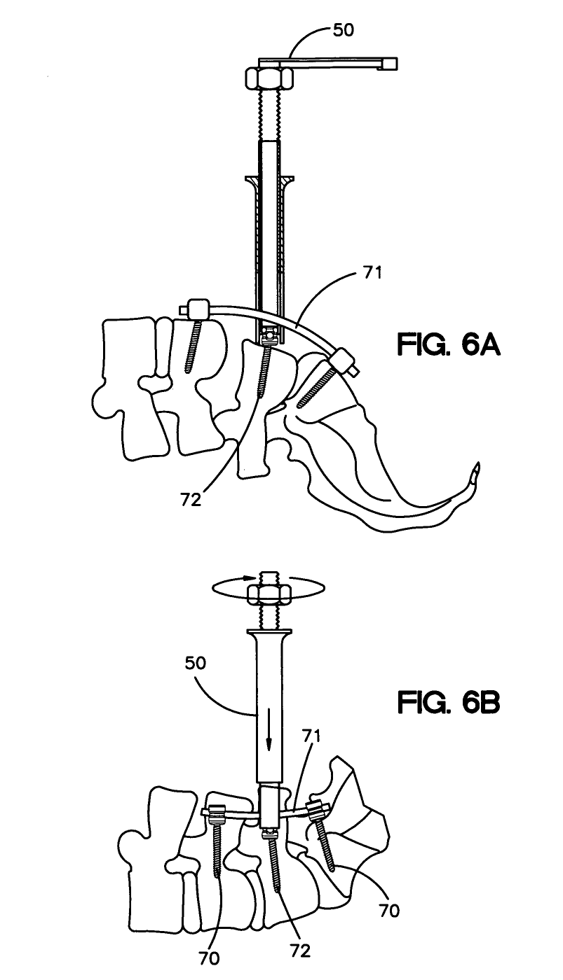

The locking screwing mechanism 40 of the interbody spacer 30 may comprise a central screw (FIG. 5A) that upon rotation may move one of the halves 31, 32 either forward or backward. In FIG. 5B, the screwing mechanism 40 may be a central rail that allows forward and backwards movement of the upper and lower halves 31, 32 and a lateral pin/rod or ratchet 41 to secure the two halves 31, 32 in position. The lateral pin 41 may project through the lateral sides 36, 37 of one of the halves 31, 32. As shown in FIG. 5B, the lateral pin is inserted in the upper halve 31 of the interbody spacer 30.

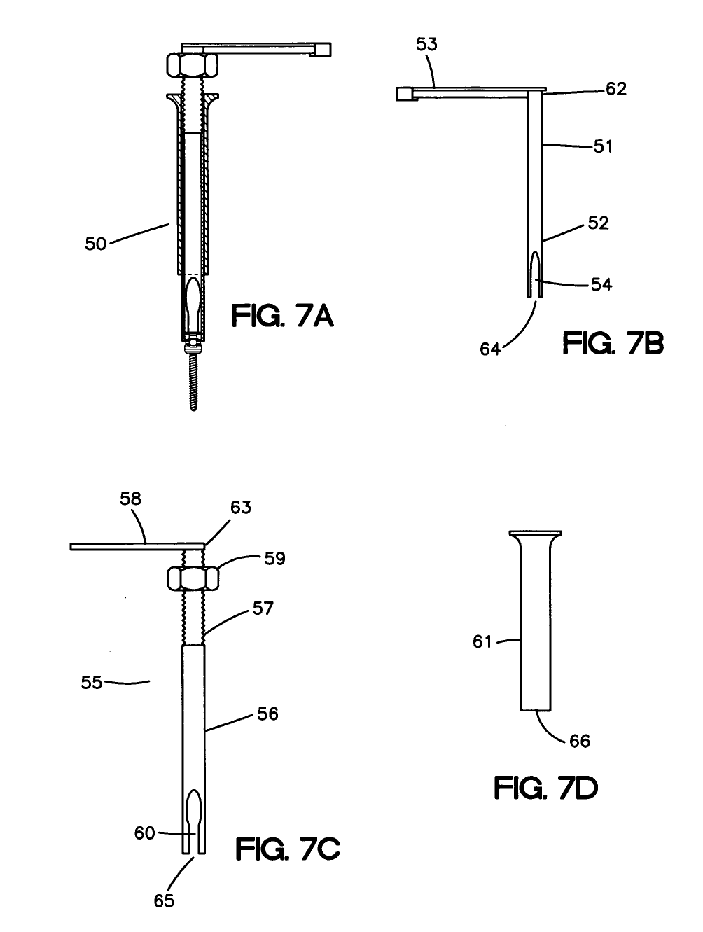

In another embodiment, as depicted in FIGS. 6A and 6B, spondylolisthesis reduction may be accomplished using pedicle screws, spondylo screws or similar 70, rods 71, repositioning instruments 50, and preassembled pedicle screws 72. In this embodiment, spondylolisthesis reduction is accomplished from the posterior.

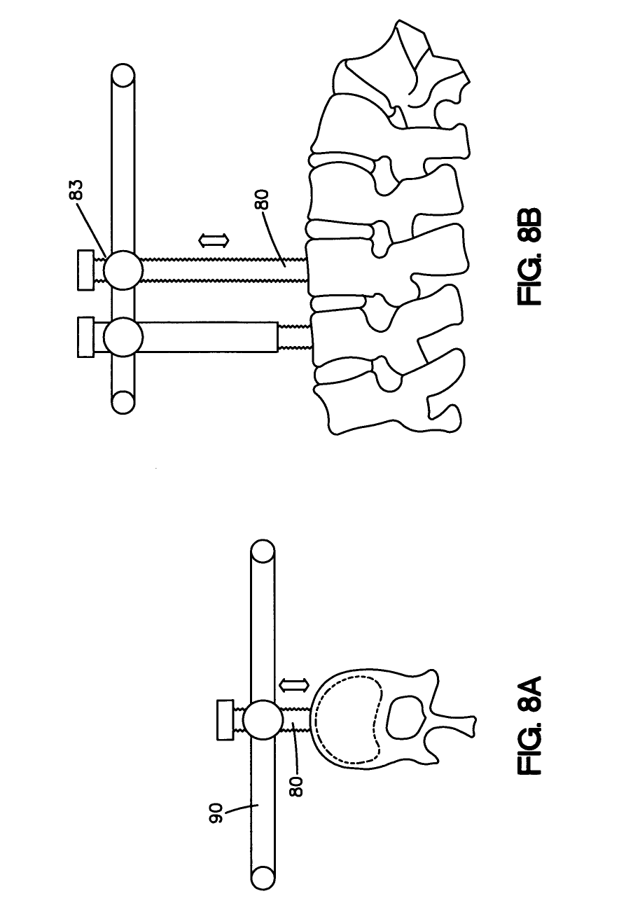

The reduction instrument 50 (FIGS. 7A-D) may include three main assemblies, an inner tube 51, a reduction sleeve 55, and a guiding tube 61. The inner tube 51 may include a linear shaft 52 having a slot 54 at the distal end 64 and a perpendicular handle 53 at its proximal end 62. The reduction sleeve 55 may also have a linear shaft 56 with a slot 60 at its distal end 65. The linear shaft 56 may have external threads 57 at the proximal end 63 about which a nut 59 is attached. The nut 59 may be used to pull a preassembled pedicle screw 72 towards a rod 71. Also attached to the proximal end 63 of the reduction sleeve 55 is a handle 58, perpendicular to the linear shaft 56. The linear shaft 56 of the reduction sleeve 55 is hollow, allowing the inner tube 51 to be inserted into the proximal end 63 of the reduction sleeve 55. The third main assembly of the reduction instrument 50 is a guiding tube 61 which fits over the reduction sleeve 55 and which tightens the instrument securely to the implant.

Pedicle screws 70 are attached to the vertebrae on either side of the displaced vertebra. One or more preassembled pedicle screws 72 are attached to the displaced vertebra. Rods 71 are inserted and locked onto the pedicle screws 70 attached to either side of the displaced vertebra. Reduction instrument 50 is placed over each preassembled pedicle screw 72. The nut 59 and reduction sleeve 55 on the reduction instrument 50 are simultaneously rotated to gradually pull the preassembled pedicle screws 72 to the rod 71 which moves the displaced vertebra. More specifically, the guiding tube 61 is moved distally as the nut 59 is rotated so that the distal end 66 of the guiding tube 61 contacts the rod which is arranged in the slot 60 of the reduction sleeve 55. Further rotation moves the reduction sleeve 55 relative to the guiding tube 61 which pulls the preassembled pedicle screw 72 and hence the vertebra upward. Once the preassembled pedicle screw 72 coincide with the rod 71, so that the rod is within a channel (not shown) in the top of the preassembled pedicle screw 72, the inner tube 51 of the reduction instrument 50 is removed and a long screwdriver with a locking cap (not shown) is inserted in the proximal end 63 of the reduction sleeve 55. The locking cap may be affixed onto the head of the preassembled pedicle screw 72, thereby securing the rod 71 to the preassembled pedicle screw 72.

The ex-situ methods for spondylolisthesis reduction allows for a minimally invasive procedure outside the wound site using adequate instruments.

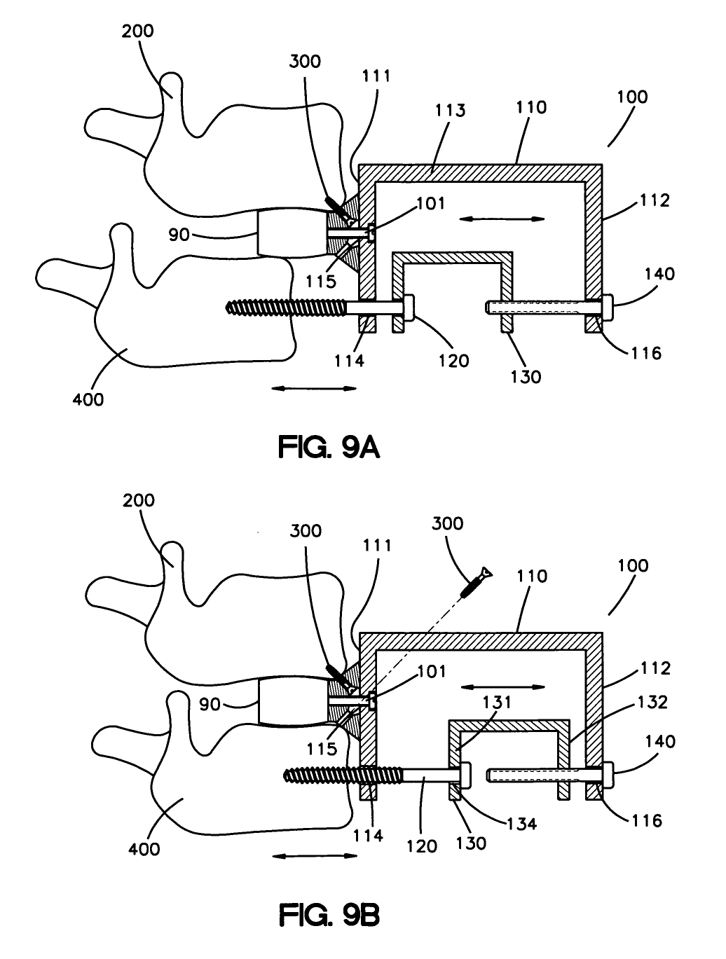

In one embodiment of the ex-situ method, depicted in FIGS. 8A and 8B, a screw 80 is inserted into the anterior part of each vertebral body at the levels to be reduced. One of the screws 80 is fixed to an external rigid element 90 (e.g. SynFrame) attached to, for example, a surgical table. The second screw 80, which is not fixed to the surgical table, is attached to an adjustable mechanism 83 (e.g. a thread member). The second screw 80 may be displaced by the adjustable mechanism 83 until the slipping distance is reduced. As noted, the adjustable mechanism 83 may be a thread member such that the thread of the second screw 80 corresponds to the thread of the adjustable mechanism forming a screw-in-screw type configuration, such that when the adjustable mechanism is rotated it pulls the displaced vertebra upwards (posterior direction).

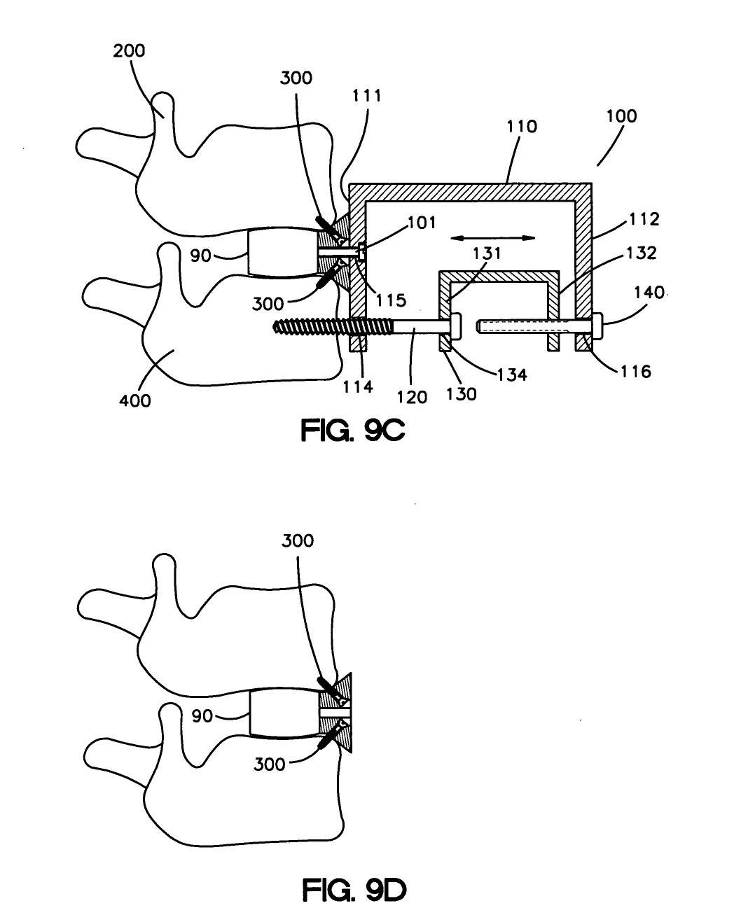

In another embodiment, as depicted in FIGS. 9A-D, spondylolisthesis reduction may be accomplished using a replacement support system 100. The replacement support system 100 may include an outer support 110, one or more bone screws 120, an inner support 130, and one or more translation screws 140.

The outer support 110 may have, for example, a U-shape with two sides 111, 112 and a connecting piece 113. One side 111 may have at least two holes 114, 115. The wall of hole 115 may have threads for engaging the threads of a screw. Whereas, the wall of hole(s) 114 is preferably smooth. The other side 112 may have at least one hole 116 whose wall is also preferably smooth.

The inner support 130 may also be U-shaped, similar to the outer support 110, with sides 131 and 132 and connecting piece 133. Both sides 131, 132 may each have at least one hole 134, 136. The wall of hole 134 is preferably smooth, whereas the wall of hole 136 preferably has threads. The inner support 130 may be smaller than the outer support 110 such that it may be positioned between sides 111 and 112.

The following describes the assembly and method of using the replacement support system 100. After having mobilized/distracted a spinal segment, a spacer 90 may be inserted between two vertebrae. The spacer 90 may be fixed to first vertebra 200 by a locking screw mechanism 300. The replacement support system 100 may be assembled such that the outer support 110 is attached to the spacer 90 by a screw 101 or other fixation device through hole 115. One or more bone screws 120 or similar fixation means may be screwed into the second vertebra 400. The one or more bone screws 120, extending through holes 114 and 134, are supported by but not affixed to the outer support 110 and inner support 130. Sides 112 and 132 are coupled through one or more translation screws 140, such that the one or more translation screws are supported by the outer support 110 but connected to the inner support 130 by corresponding threads on the screw and wall of hole 136. Rotation of the one or more translation screws 140 allow for movement of the inner support 130 with respect to the outer support 110. Movement can consist of either pulling or pushing back one of the second vertebra.

After the replacement support system 100 has been installed onto the vertebrae, the second vertebra 400 can be pulled or pushed back by rotating the one or more translation screws 140 until the first and second vertebrae are aligned such that the spacer 90 may be fixed onto the second vertebra 400. Following the repositioning procedure, one or more screws 300 may be inserted into the spacer 90 and second vertebra 400, fixing the spacer 90 to the second vertebra 400 (FIGS. 9B and 9C). Once the one or more screws 300 fixing the spacer 90 and second vertebra 400 are in place, the replacement support system 100 can be removed (FIG. 9D).

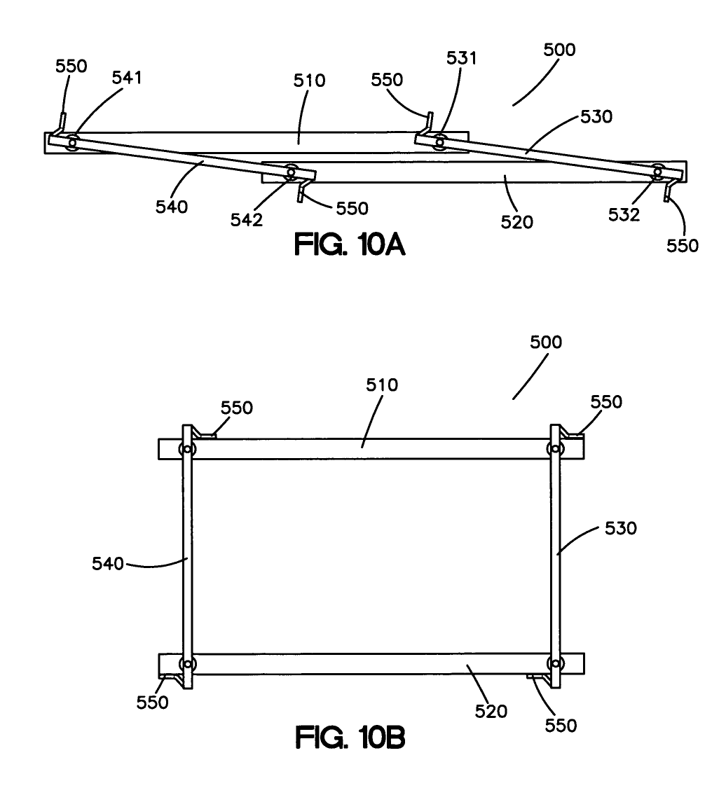

In another embodiment, a spacer 500 may be expanded allowing for repositioning and distracting of vertebrae. The spacer 500 may comprise an upper and lower spacer plates 510, 520. The spacer plates 510, 520 may have the shape and footprint similar to existing interbody fusion implant geometries. The spacer plates 510, 520 may be connected by two or more bars 530, 540. The bars 530, 540 may be connected to the spacer plates 510, 520 by a hinge, joint, or some similar connecting means 531, 532, 541, 542. As shown in FIG. 10A, the spacer 500 is in an unexpanded form, in which the bars 530, 540 are substantially parallel with the spacer plates 510, 520. In FIG. 10B, the spacer 500 is in an expanded form, where the spacer plates 510, 520 are positioned further apart from one another and the bars 530, 540 are substantially perpendicular to the spacer plates 510, 520. The angle of the bars 530, 540 with respect to the spacer plates 510, 520 may be determined/chosen according to the amount of repositioning and distraction needed. A fixation mechanism (not shown) within the joints/hinges maintain the angle of the bars 530, 540 with respect to the spacer plates 510, 520, stabilizing the structure of the spacer 500 and ensuring that the reposition of the vertebrae does not move subsequently.

The spacer 500, in its unexpanded form, may be inserted between two vertebrae (not shown) exhibiting spondylolisthesis. The upper and lower spacer plates 510, 520 may be fixed to the vertebrae with screws (not shown). After the upper and lower spacer plates 510, 520 have been fixed to the vertebrae, the spinal segment is repositioned and distracted by expanding the spacer 500 such that the space between the vertebrae is increased and simultaneously repositioning the vertebrae until they are aligned. The bars 530, 540 and fixation mechanisms ensure the spacer 500 maintains its expanded form, thereby stabilizing the spinal segment. The void created between the spacer plates 510, 520 may be filled with autologous bone or bone substitute to allow for fusion between the upper and lower vertebrae. The lateral and posterior parts of the spacer 500 may be surrounded by a membrane, initially fixed to the spacer plates 510, 520, to avoid the autologous bone or bone substitute from escaping.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

It will be appreciated by those skilled in the art that various modifications and alterations of the invention can be made without departing from the broad scope of the appended claims. Some of these have been discussed above and others will be apparent to those skilled in the art.

1. A method of performing spondylolisthesis reduction, the method comprising the steps of:
inserting an interbody spacer between two vertebrae;
attaching a reduction plate to the two vertebrae by at least two screws, wherein the reduction plate includes an upper and lower borehole, and the at least one screw uses a stable plate-screw connection, and at least another screw is fixed to the other vertebra is a non-locking screw; and
driving the non-locking screw to reduce the vertebral slippage distance.

2. The method of claim 1, wherein the reduction plate is straight.

3. The method of claim 1, wherein the reduction plate is curved, in a pre-stressed condition.

4. The method of claim 1, wherein the reduction plate can be adjusted intraoperatively for anatomical alignment of the vertebrae.

5. The method of claim 1, wherein the reduction plate includes a central borehole for securing the interbody spacer to the reduction plate by a screw.

6. A method of performing spondylolisthesis reduction, the method comprising the steps of:
inserting an interbody spacer between a first and second vertebrae;
attaching the interbody spacer to one of the first vertebrae;
attaching an outer support to the interbody spacer;
securing at least one bone screw into the second vertebra, wherein the at least one screw is supported and not fixed to an inner support and the outer support;
threading at least one translation screw to the inner support, wherein the at least one translation screw is supported by the outer support;
rotating the translation screw to reduce the vertebral slippage distance;
inserting at least one screw into the second vertebra and interbody spacer, wherein the interbody spacer is stably fixed to the other vertebra; and
removing the outer support.

7. The method of claim 6, wherein the inner support and outer support are U-shaped.

8. The method of claim 6, wherein reduction of the vertebral slippage occurs by pulling the second vertebra.

9. The method of claim 6, wherein reduction of the vertebral slippage occurs by pushing the second vertebra.

10. A method of performing spondylolisthesis reduction, the method comprising the steps of:
inserting an interbody spacer between two vertebrae, wherein the interbody spacer consists of a first member, a second member and an adjusting mechanism;
attaching the first member and second member to the vertebrae by locking screws; and
manipulating the adjusting mechanism such that the first member and second member move laterally with respect to each other, thereby aligning the vertebrae.

11. A method of performing spondylolisthesis reduction on a first and second vertebrae, the method comprising the steps of:
inserting at least one screw into the first vertebrae,
fixing at least one screw to the second vertebra and to an external rigid element; and
using an adjustable mechanism to adjust the screw inserted into the second vertebra until a slippage distance of the second vertebra is reduced,
wherein the method is performed externally from an incision area.
